(19) United States
(12) Patent Application Publication
(10) Pub. No.: US 2021/0068676 A1
(43) Pub. Date: Mar. 11, 2021

(54) METHOD AND SYSTEM FOR AUTOMATICALLY GENERATING AND ANALYZING FULLY QUANTITATIVE PIXEL-WISE MYOCARDIAL BLOOD FLOW AND MYOCARDIAL PERFUSION RESERVE MAPS TO DETECT ISCHEMIC HEART DISEASE USING CARDIAC PERFUSION MAGNETIC RESONANCE IMAGING

(71) Applicants: The United States of America, Department of Health and Human Services, National Institutes of Health, Bethesda, MD (US); Circle Cardiovascular Imaging Inc., Calgary (CA)

(72) Inventors: Li-Yueh HSU, Vienna, VA (US); Matthew JACOBS, Riverdale, MD (US); Mitchel BENOVOY, Montréal, Québec (CA); Andrew ARAI, Kensington, MD (US)

(21) Appl. No.: 17/046,193

(22) PCT Filed: May 17, 2019
(86) PCT No.: PCT/IB2019/054112

§ 371 Date: Oct. 8, 2020

Related U.S. Application Data
(60) Provisional application No. 62/680,185, filed on Jun. 4, 2018.
(60) Provisional application No. 62/672,787, filed on May 17, 2018.

(51) Int. Cl. A61B 5/026; A61B 5/055; G01R 33/56; G01R 33/563

(52) U.S. Cl. CPC A61B 5/0263; G01R 33/56366; G01R 33/5608; A61B 5/055

(57) ABSTRACT

A computer-implemented method for automatically generating a fully quantitative myocardial blood flow map, comprising: receiving myocardial perfusion magnetic resonance imaging (MRI) images and arterial input function (AIF) MRI images; correcting a motion of a heart in the myocardial perfusion MRI images and the AIF MRI images, thereby obtaining motion corrected myocardial perfusion MRI images and motion corrected AIF images; correcting an intensity of the motion corrected myocardial perfusion MRI images and an intensity of the motion corrected AIF images, thereby obtaining surface coil intensity corrected MRI images and surface coil intensity corrected AIF images; using the surface coil intensity corrected MRI images and the surface coil intensity corrected AIF images, determining time-signal intensity characteristics and segmenting a left ventricle myocardial tissue region; and generating the myocardial blood flow map using the motion corrected myocardial perfusion MRI images, the left ventricle myocardial tissue region segmentation and the time-signal intensity characteristics.

### STATEMENT OF GOVERNMENT INTEREST

This invention was made with Government support under project number 1ZIAHL006137-01 through 1ZIAHL006137-08 by the National Institutes of Health, National Heart, Lung, and Blood Institute, and under contract numbers HHSN268201600299A and HHSN268201700377A. The Government has certain rights in the invention.

### TECHNICAL FIELD

The present invention relates to the field of myocardial blood flow analysis and detection of ischemic heart disease using cardiac magnetic resonance imaging perfusion image series, and more particularly to the automatic fully quantitative myocardial blood flow analysis and cardiac disease detection via myocardial blood flow maps, their derived features, and automatic classification.

### BACKGROUND

No system allowing fully automatic, fully quantitative myocardial perfusion analysis of myocardial perfusion magnetic resonance images exists. Consequently, data for fully quantitative blood flow (MBF) and myocardial perfusion reserve (MPR) analysis must be manually performed, which is both laborious and time consuming.

Therefore, there is a need for a method and system for automatically generating fully quantitative MBF and MPR maps and using these maps to extract features to perform automatic cardiac disease detection and diagnosis.

### SUMMARY

According to a first broad aspect, there is provided a computer-implemented method for automatically generating a fully quantitative myocardial blood flow map, comprising: receiving myocardial perfusion magnetic resonance imaging (MRI) images and arterial input function (AIF) MRI images; correcting a motion of a heart in the myocardial perfusion MRI images and the AIF MRI images, thereby obtaining motion corrected myocardial perfusion MRI images and motion corrected AIF images; correcting an intensity of the motion corrected myocardial perfusion MRI images and an intensity of the motion corrected AIF images, thereby obtaining surface coil intensity corrected MRI images and surface coil intensity corrected AIF images; using the surface coil intensity corrected MRI images and the surface coil intensity corrected AIF images, determining time-signal intensity characteristics and segmenting a left ventricle myocardial tissue region; generating the myocardial blood flow map using the motion corrected myocardial perfusion MRI images, the left ventricle myocardial tissue region segmentation and the time-signal intensity characteristics; and outputting the myocardial blood flow map.

In one embodiment, the step of correcting the motion of the heart comprises detecting the motion of the heart in the myocardial perfusion MRI images and the AIF MRI images.

In one embodiment, the step of detecting the motion of the heart comprises generating a first copy of the myocardial perfusion MRI images and a second copy of the AIF MRI images, and rescaling the first and second copies, thereby obtaining a rescaled copy of myocardial perfusion MRI images and a rescaled copy of AIF MRI images.

In one embodiment, the step of detecting the motion of the heart comprises performing a non-rigid displacement estimation on the rescaled copy of. myocardial perfusion MRI images and a rescaled copy of AIF MRI images.

In one embodiment, the non-rigid displacement estimation comprises a large displacement optical flow estimation.

In one embodiment, the method further comprises identifying a reference frame for each one of the rescaled copy of myocardial perfusion MRI images and the rescaled copy of AIF MRI images.

In one embodiment, the method further comprises denoising the rescaled copy of myocardial perfusion MRI images and the rescaled copy of AIF MRI images prior to identifying the reference frame.

In one embodiment, the step of denoising is performed using a principle component analysis method.

In one embodiment, the step of correcting the motion of the heart comprises registering the myocardial perfusion MRI images and the AIF MRI images to the reference frame, thereby obtaining the motion corrected myocardial perfusion MRI images and the motion corrected AIF images.

In one embodiment, the step of registering is performed using an interpolation warping method.

In one embodiment, the interpolation warping method comprises a 2D bi-cubic interpolation warping method.

In one embodiment, the method further comprises recovering an original signal intensity distribution for the motion corrected myocardial perfusion MRI images and the motion corrected AIF images.

In one embodiment, the step of correcting the intensity comprises: estimating a signal intensity bias field; and correcting the motion corrected myocardial perfusion MRI images and the motion corrected AIF images using the a signal intensity bias field, thereby obtaining the surface coil intensity corrected MRI images and the surface coil intensity corrected AIF images.

In one embodiment, the step of estimating the signal intensity bias field is performed using a fitting method.

In one embodiment, the fitting method comprises a fifth-order 2D polynomial least square fitting method.

In one embodiment, the step of determining time-signal intensity characteristics and segmenting a left ventricle myocardial tissue region comprises identifying a left ventricle and a right ventricle

In one embodiment, the step of identifying the left ventricle and the right ventricle comprises: determining candidate ventricle regions within the surface coil intensity corrected MRI images and the surface coil intensity corrected AIF images; using a similarity check method to regroup particular ones of the candidate ventricle regions to obtain two ventricle regions; and using a linear voting scheme to assign a first one of the two ventricle regions to the left ventricle and a second one of the two ventricle regions to the right ventricle.

In one embodiment, the linear voting scheme is based on at least one of: a distance to an image center, a distance to previously selected candidate regions, a size of a region, a signal intensity upslope, a peak value (PV), a time to peak (TTP), a full width at half maximum (FWHM), and an M-value.

In one embodiment, the step of determining time-signal intensity characteristics comprises determining a myocardial time-signal intensity curve and an AIF time-signal intensity curve.

In one embodiment, the step of generating the myocardial blood flow map is performed using a pixel-wise deconvolution method.

In one embodiment, the step of receiving further comprises receiving proton density (PD) images and non-linearly registering the PD images to the myocardial perfusion MRI images and the AIF MRI images.

According to a second broad aspect, there is provided a system for automatically generating a fully quantitative myocardial blood flow map, comprising: a motion correction unit for receiving myocardial perfusion magnetic resonance imaging (MRI) images and arterial input function (AIF) MRI images and correcting a motion of a heart in the myocardial perfusion MRI images and the AIF MRI images in order to obtain motion corrected myocardial perfusion MRI images and motion corrected AIF images; an intensity correction unit for correcting an intensity of the motion corrected myocardial perfusion MRI images and an intensity of the motion corrected AIF images in order to obtain surface coil intensity corrected MRI images and surface coil intensity corrected AIF images; an analyzer unit for determining time-signal intensity characteristics and segmenting a left ventricle myocardial tissue region using the surface coil intensity corrected MRI images and the surface coil intensity corrected AIF images; and a map generator for generating the myocardial blood flow map using the motion corrected myocardial perfusion MRI images, the left ventricle myocardial tissue region segmentation and the time-signal intensity characteristics, and outputting the myocardial blood flow map.

In one embodiment, the motion correction unit is configured for detecting the motion of the heart in the myocardial perfusion MRI images and the AIF MRI images.

In one embodiment, the motion correction unit is configured for generating a first copy of the myocardial perfusion MRI images and a second copy of the AIF MRI images, and rescaling the first and second copies, thereby obtaining a rescaled copy of myocardial perfusion MRI images and a rescaled copy of AIF MRI images.

In one embodiment, the motion correction unit is configured for performing a non-rigid displacement estimation on the rescaled copy of. myocardial perfusion MRI images and a rescaled copy of AIF MRI images.

In one embodiment, the non-rigid displacement estimation comprises a large displacement optical flow estimation.

In one embodiment, the motion correction unit is further configured for identifying a reference frame for each one of the rescaled copy of myocardial perfusion MRI images and the rescaled copy of AIF MRI images.

In one embodiment, the motion correction unit is further configured for denoising the rescaled copy of myocardial perfusion MRI images and the rescaled copy of AIF MRI images prior to identifying the reference frame.

In one embodiment, said denoising is performed using a principle component analysis method.

In one embodiment, the motion correction unit is configured for registering the myocardial perfusion MRI images and the AIF MRI images to the reference frame to obtain the motion corrected myocardial perfusion MRI images and the motion corrected AIF images.

In one embodiment, said registering is performed using an interpolation warping method.

In one embodiment, the interpolation warping method comprises a 2D bi-cubic interpolation warping method.

In one embodiment, the motion correction unit is further configured for recovering an original signal intensity distribution for the motion corrected myocardial perfusion MRI images and the motion corrected AIF images.

In one embodiment, the intensity correction unit is configured for: estimating a signal intensity bias field; and correcting the motion corrected myocardial perfusion MRI images and the motion corrected AIF images using the a signal intensity bias field, thereby obtaining the surface coil intensity corrected MRI images and the surface coil intensity corrected AIF images.

In one embodiment, said estimating the signal intensity bias field is performed using a fitting method.

In one embodiment, the fitting method comprises a fifth-order 2D polynomial least square fitting method.

In one embodiment, the analyzer unit is configured for identifying a left ventricle and a right ventricle

In one embodiment, said identifying the left ventricle and the right ventricle comprises: determining candidate ventricle regions within the surface coil intensity corrected MRI images and the surface coil intensity corrected AIF images; using a similarity check method to regroup particular ones of the candidate ventricle regions to obtain two ventricle regions; and using a linear voting scheme to assign a first one of the two ventricle regions to the left ventricle and a second one of the two ventricle regions to the right ventricle.

In one embodiment, the linear voting scheme is based on at least one of: a distance to an image center, a distance to previously selected candidate regions, a size of a region, a signal intensity upslope, a peak value (PV), a time to peak (TTP), a full width at half maximum (FWHM), and an M-value.

In one embodiment, the analyzer unit is configured for determining a myocardial time-signal intensity curve and an AIF time-signal intensity curve.

In one embodiment, the map generator is configured for generating the myocardial blood flow map using a pixel-wise deconvolution method.

According to another broad aspect, there is provided a computer-implemented method for automatically detecting and diagnosing heart disease, comprising: receiving myocardial perfusion magnetic resonance imaging (MRI) images, time-signal intensity curves and rest and stress myocardial blood flow maps; determining a myocardial perfusion reserve (MPR) map and segmented regions of interest of a left ventricle using the rest and stress myocardial blood flow maps and the myocardial perfusion MRI images; extracting features of interest from the segmented regions of interest, the MPR maps, the time-signal intensity curves and the rest and stress myocardial blood flow maps; and automatically classifying the features of interest, thereby obtaining a classification output; and outputting the classification output, the classification output depicting normal versus abnormal myocardial regions and corresponding coronary territories.

In one embodiment, the step of determining the segmented regions of interest of the left ventricle is based on an analysis of pixel dynamics.

In one embodiment, the step of determining the MPR map is performed using a non-rigid registration of the rest myocardial blood flow map to the stress myocardial blood flow map.

In one embodiment, the classification output comprises at least one of an imaging quality assurance factor, symbols and markers labelling a location and a size of suspected myocardial lesions, an anatomical mapping of perfusion defect regions to a coronary artery anatomy, patterns of perfusion defect and a diagnostic report.

In one embodiment, the imaging quality assurance factor comprises one of a heart rate, an RR interval during electrocardiogram gating, a vasodilator systematic response and a signal intensity linearity measurement.

According a further broad aspect, there is provided a system for automatically detecting and diagnosing heart disease, comprising:
a region determining unit for receiving myocardial perfusion magnetic resonance imaging (MRI) images, time-signal intensity curves, and rest and stress myocardial blood flow maps, and determining a myocardial perfusion reserve (MPR) map and segmented regions of interest of a left ventricle using the rest and stress myocardial blood flow maps; a feature extraction unit for extracting features of interest from the segmented regions of interest, the MPR map, the time-signal intensity curves, and the rest and stress myocardial blood flow maps; and a classification unit for classifying the extracted features of interest to obtain a classification output and outputting the classification output to depict normal versus abnormal myocardial regions and corresponding coronary territories.

In one embodiment, the region determining unit is configured for determining the segmented regions of interest of the left ventricle is based on an analysis of pixel dynamics.

In one embodiment, the feature extraction unit is configured for determining the MPR map using a non-rigid registration of the rest myocardial blood flow map to the stress myocardial blood flow map.

In one embodiment, the classification output comprises at least one of an imaging quality assurance factor, symbols and markers labelling a location and a size of suspected myocardial lesions, an anatomical mapping of perfusion defect regions to a coronary artery anatomy, patterns of perfusion defect and a diagnostic report.

In one embodiment, the imaging quality assurance factor comprises one of a heart rate, an RR interval during electrocardiogram gating, a vasodilator systematic response and a signal intensity linearity measurement.

According to still another broad aspect, there is provided a computer-implemented method for analyzing a myocardial blood flow, comprising: receiving myocardial perfusion magnetic resonance imaging (MRI) images, time-signal intensity curves and rest and stress myocardial blood flow maps; determining a myocardial perfusion reserve (MPR) map and segmented regions of interest of a left ventricle using the rest and stress myocardial blood flow maps and the myocardial perfusion MRI images; extracting features of interest from the segmented regions of interest, the MPR maps, the time-signal intensity curves and the rest and stress myocardial blood flow maps; and outputting the features of interest.

According to still a further broad aspect, there is provided a system for analyzing a myocardial blood flow, comprising: a region determining unit for receiving myocardial perfusion magnetic resonance imaging (MRI) images, time-signal intensity curves and rest and stress myocardial blood flow maps, and determining a myocardial perfusion reserve (MPR) map and segmented regions of interest of a left ventricle using the rest and stress myocardial blood flow maps; and a feature extraction unit for extracting features of interest from the segmented regions of interest, the MPR maps that were measured globally in the entire left ventricle (LV) myocardium and/or within specific regions of interest, the time-signal intensity curves, the pixel-wise and sector-wise perfusion indices, the size, the location, the texture, the pattern, the severity, the grading of the lesion (perfusion defect) within the myocardium, and the rest and stress myocardial blood flow maps and outputting the features of interest.

It should be understood that the expression “myocardial blood flow pixel map” (also referred to as “myocardial blood flow map” hereinafter) refers to a fully quantitative myocardial blood flow map which shows the amount of blood perfusing (irrigating) the heart muscle tissue at each pixel in the images in absolute and/or relative units that are expressed in physiological units (e.g. ml/g/min, gram, second) or as arbitrary units (e.g. signal intensity, rate, or percentage). A fully quantitative myocardial blood flow map shows the amount of blood going through the heart tissue, which is fed by the coronary arteries. A fully quantitative myocardial blood flow map does not only show the amount of blood going through the muscle, but depending on the location of areas where blood is not going through enough, a fully quantitative myocardial blood flow map may indicate which branch of the coronaries is blocked.

### BRIEF DESCRIPTION OF THE DRAWINGS

Further features and advantages of the present invention will become apparent from the following detailed description, taken in combination with the appended drawings, in which:

FIG. 1 is a flow chart of a method for generating a myocardial blood flow pixel map, in accordance with an embodiment;

FIGS. 2a and 2b present exemplary pixel-wise myocardial blood flow maps for stress series and pixel-wise myocardial blood flow maps for rest series, respectively, at three different slice locations;

FIG. 3 illustrates a large displacement optical flow, in accordance with an embodiment;

FIG. 4 illustrates a PD-to-T1 registration, in accordance with an embodiment;

FIGS. 5a-5d illustrate a PD image, an intensity sampling of PD image, an estimated 2D bias field and a corrected perfusion image, respectively, in accordance with an embodiment;

FIG. 6 illustrates a process for time-signal intensity characteristics, in accordance with an embodiment;

FIG. 7 illustrates a AIF timing point detection, in accordance with an embodiment;

FIG. 8 illustrates a pixel-wise deconvolution to obtain a myocardial blood flow pixel map, in accordance with an embodiment; and

FIG. 9 is a block diagram of a system for generating a myocardial blood flow pixel map, in accordance with an embodiment;

FIG. 10 is a block diagram of a processing module adapted to execute at least some of the steps of the method of FIG. 1, in accordance with an embodiment;

FIG. 11 is a flow chart of a method for extracting features of interests from the myocardial blood flow pixel map generated by the method of FIG. 1, in accordance with an embodiment;

FIG. 12a illustrates a raw image of a heart, in accordance with an embodiment;

FIGS. 12b and 12c illustrate exemplary automatic region-of-interest segmentation of the raw image of FIG. 12a into the right and left ventricles, and the left ventricle myocardium, respectively.

FIGS. 13a, 13b and 13c illustrate exemplary sector-wise polar plots of myocardial blood flow for a stress series, a rest series, and the myocardial perfusion reserve, respectively;

FIG. 14 is a block diagram of a system for extracting features of interests from the myocardial blood flow pixel map generated by the system of FIG. 8, in accordance with an embodiment; and

FIG. 15 is a block diagram of a processing module adapted to execute at least some of the steps of the method of FIG. 10, in accordance with an embodiment.

It will be noted that throughout the appended drawings, like features are identified by like reference numerals.

### DETAILED DESCRIPTION

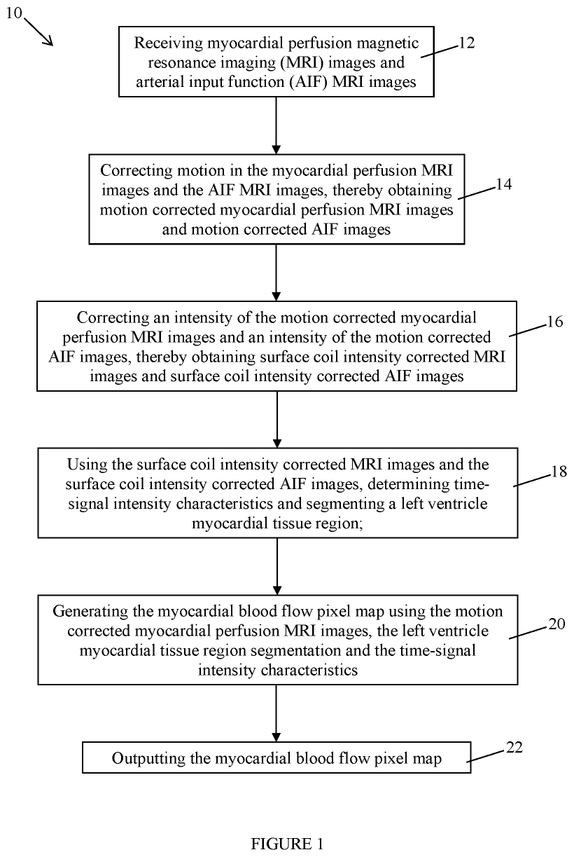

FIG. 1 illustrates a computer-implemented method 10 for generating a myocardial blood flow pixel map. It should be understood that the method 10 is implemented by a computer machine comprising at least one processor or processing unit, a memory and communication means, the memory having stored thereon statements and instructions that when executed by the processor perform the steps 12-22 of the method 10.

At step 12, a series of myocardial perfusion magnetic resonance imaging (MRI) images and a series of arterial input function (AIF) MRI images are received. It should be understood that the myocardial perfusion MRI images and the AIF MRI images are taken either substantially concurrently or sequentially. The MRI images may comprise fast low-angle shot (FLASH) MRI images, steady-state free-precession (FISP) MRI images or any other type of MRI images suitable for perfusion studies. The acquisition paradigm may be either free-breathing, breath-hold under dual-bolus or dual-sequence methods.

At step 14, motion correction is performed for the myocardial perfusion MRI images and the AIF MRI images in order to correct for motion of the heart, thereby obtaining motion corrected myocardial perfusion MRI images and motion corrected AIF images, as described below.

At step 16, intensity correction is performed for the motion corrected myocardial perfusion MRI images and the motion corrected AIF images to improve the images' intensity homogeneity of magnetic resonance imaging, thereby obtaining surface coil intensity corrected perfusion MRI images and surface coil intensity corrected AIF images, as described below.

At step 18, using the surface coil intensity corrected perfusion MRI images and the surface coil intensity corrected AIF images, time-signal intensity characteristics/metrics are determined and the left ventricle myocardial tissue region is identified and segmented in both the AIF and the myocardial perfusion MRI images, as described in greater detail below.

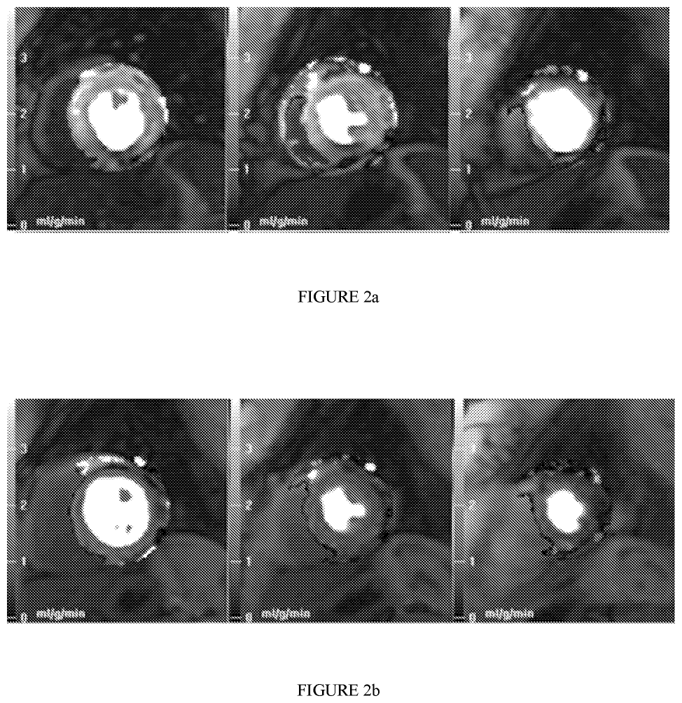

At step 20, a myocardial blood flow pixel map is generated using the motion corrected myocardial perfusion MRI images, the left ventricle myocardial tissue region segmentation and the time-signal intensity characteristics, as described below. FIG. 2a illustrate an exemplary myocardial blood flow pixel map for stress series at three different slice locations while FIG. 2b illustrates an exemplary myocardial blood flow pixel map for rest series, at three different slice locations.

At step 22, the myocardial blood flow pixel map is outputted. In one embodiment, the myocardial blood flow pixel map is stored in memory. In the same or another embodiment, the myocardial blood flow pixel map is transmitted to a computer machine. In one embodiment, the myocardial blood flow pixel map is displayed on a display unit.

In one embodiment, the step 12 of the method 10 further comprises receiving proton density (PD) images. In this case, the method 10 further comprise a step of non-linearly registering the PD images to the myocardial perfusion and AIF MRI images, as described below, and the step 18 is performed further using the PD images registered to the myocardial perfusion MRI images.

In one embodiment, the series of series of AIF MRI images and the series of myocardial perfusion MRI images comprise a series of stress AIF MRI images and a series of stress myocardial perfusion MRI images, respectively. In this case, a series of stress AIF MRI images and a series of stress myocardial perfusion MRI images are received at step 12 and myocardial blood flow maps of stress are generated.

In another embodiment, the series of series of AIF MRI images and the series of myocardial perfusion MRI images comprise a series of rest AIF MRI images and a series of rest myocardial perfusion MRI images, respectively. In this case, a series of rest AIF MRI images and a series of rest myocardial perfusion MRI images are received at step 12 and myocardial blood flow maps of rest are generated.

In a further embodiment, the series of series of AIF MRI images and the series of myocardial perfusion MRI images comprise a series of stress and rest AIF MRI images and a series of stress and rest myocardial perfusion MRI images. In this case, a series of stress and rest AIF MRI images and a series of stress and rest myocardial perfusion MRI images are received at step 12, and both myocardial blood flow maps of stress and myocardial blood flow maps of rest are generated.

In the following there is described one exemplary embodiment of the method 10.

First series or sequences of raw images of a heart are received and the received images are processed to correct for motion of the heart. The series of received raw images comprise stress and/or rest AIF MRI series and stress and/or rest myocardial perfusion MRI series with or without PD images.

In order to perform the motion correction in the series of images, the method uses post-hoc interpolation warping following non-rigid displacement estimation based on an optical flow formulation specifically amenable to perfusion series dynamics. In one embodiment, the present method offers robustness to breathing paradigms, an ability to register cardiac perfusion image series with varying characteristics, and a compatibility with the processing of auxiliary series such as PD images and independent AIF acquisitions used to facilitate fully quantitative myocardial perfusion analysis. The implementation also features automatic reference frame and PD image detection and a multithreaded architecture to improve processing speed.

The raw images, i.e. the myocardial perfusion MRI images, the AIF MRI images and the PD images, if any, are first pre-processed. A copy such as a proxy copy of the raw image series is generated and used to estimate the motion, while the original raw series is used exclusively at the later registration stages during geometrical transformations. In order to standardize processing parameters and optimize computation time, the proxy images are first rescaled to a smaller size and quantized to a lower dynamic range. As the image warping following motion estimation uses the original raw images, there is no reduction in image quality besides the expected smoothing caused by the interpolation. It should be understood that the pre-processing of the raw images may be omitted.

Then a reference myocardial perfusion MRI image or frame, a reference AIF MRI image or frame and a PD image or frame are detected from the copies of the raw images A reference frame/image is used as an initial target to which all other images will be registered. In one embodiment, the optimal reference frame is identified automatically in the late enhancement period of the series. This optimal reference frame ρ is automatically detected by first denoising the proxy series using principle component analysis (PCA) decomposition with high variance retention to reduce transient image artifacts and noise, in addition to smoothing out structural movement across the proxy series. Sequential neighbors in a reverse order of the PCA-reduced series are then cross-correlated together until a large correlation coefficient drop is detected with differential analysis, which ostensibly corresponds to the flush-out of contrast in the left ventricle (LV). The PD frames preceding the myocardial perfusion and AIF series are automatically detected in a similar way by identifying the large correlation variation corresponding to the PD to non-PD image transition.

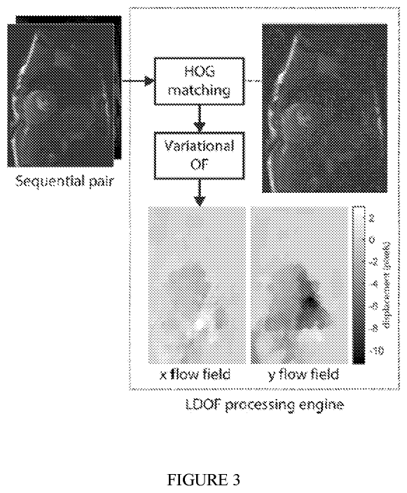

The next step consists in a large displacement optical flow (OF) motion estimation. Non-rigid deformation maps between pairwise sequential images are computed in the myocardial perfusion and AIF MRI series using an OF formulation robust to large displacement, i.e. a large displacement optical flow (LDOF). This characteristic may allow for accommodating the wide range of motion found in the myocardial perfusion MRI series and the fast ventricular bolus arrivals without causing motion estimation artifacts. As illustrated in FIG. 3, the LDOF formulation couples discrete feature points tracking with a continuous variational optical flow step in a coarse-to-fine optimization scheme. Given two images I₁, I₂ to be aligned and a two-dimensional point x in the image domain ω:x=(x, y)^{T}, the optical flow field w:=(u, v)^{T} is given by the pixel intensity energy term:

Eᵢₙₜ(w)=∫_{ω}ψ(|I₂(x+w(x))−I₁(x)|²)dx Eq. 1

where ψ is a robust function to mitigate occlusions.

Equation 1 penalizes deviations from the assumption that corresponding points should have the same intensity, but this is seldom encountered in practice so a gradient (∇) constrain invariant to additive illumination is defined:

E_{grad}(w)=∫_{ω},ψ(|∇I₂(x+w(x))−∇I₁(x)|²)dx Eq. 2

In one embodiment, Equations 1 and 2 may match relatively weak features (intensity and gradients), and may result in non-unique solutions. A regularization term applied to the computed flow fields may thus be introduced:

Eₛₘₒₒₜₕ(w)=∫_{ω}ψ(|∇u(x)|²+|∇v(x)|²)dx Eq. 3

Hence, the variational optical flow model is given by:

E(w)=Eᵢₙₜ(w)+γE_{grad}(w)+αEₛₘₒₒₜₕ(w) Eq. 4

where α, γ are tuning parameters.

Large displacement estimation is achieved by adding the landmark correspondence energy term, as follows:

E_{match}(w)=∫δ(x)τ(x)ψ(|w(x)−w₁(x)|)dx Eq. 5

where w₁(x) is a correspondence vector constructed by matching a given point x, δ(x) is a binary variable indicating the presence of a match, and τ is a matching score related to the distance between matches descriptor vectors. The task of discrete descriptor matching can be formulated in a continuous approach to make it compatible with the continuous variation model:

E_{desc}(w)=∫δ(x)|f₂(x+w₁(x))−f₁(x)|²dx Eq. 6

where f₁(x), f₂(x) are the fields of descriptor vectors in I₁, I₂.

The final LDOF model can thus be represented as follows:

E(w)=Eᵢₙₜ(w)+γE_{grad}(w)+αEₛₘₒₒₜₕ(w)βE_{match}(w,w₁)+E_{desc}(w₁) Eq. 7

The Histogram of Oriented Gradients (HOG) tracking component may handle large displacements of arbitrarily moving anatomical structures, while the a variational computation module may estimate dense flow fields to measure relatively subtle deformations such as the elastic motion of the myocardial wall at sub-pixel accuracy. The LDOF method is controlled with three primary parameters: α, i.e. the smoothness of the flow fields; β, i.e. the weight of the descriptor matching term; and γ, i.e. the weight of the gradient consistency term. These parameters are set to optimized values (e.g. small α and large β and γ) that may be automatically learned from a small subset of patients using a grid-search approach for example.

In one embodiment, instead of immediately registering the current frame before computing the subsequent motion, the stepwise deformation fields [fx, fy] of each myocardial perfusion and AIF MRI image pair are saved for warping at a later stage. In one embodiment and in comparison to methods that immediately register all images to a reference frame or to consecutively registered images, this post-hoc approach may avoid successive smoothing and improving the displacement estimation between adjacent frames. This approach may be well suited for cardiac perfusion series that have large motion events due to incomplete patient breath holding or high tissue contrast changes during late perfusion. Furthermore, as the estimation of a given flow field does not depend on pre-registered frames, the motion corrected (MOCO) pipeline may be designed as a parallel processing architecture where groups of neighboring image pairs are handled by multiple LDOF processing engines and executed by independent CPU threads.

Following motion estimation on every sequential image pair, all myocardial perfusion and AIF MRI images are registered to p using 2D bi-cubic interpolation warping with the cumulative displacements computed from the stepwise estimates. This procedure fully resolves the pixel-wise [x, y] excursions from ρ to a given image frame t:

$$[ {F}_{t}^{x} {F}_{t}^{y} ] = \{ \begin{matrix}{\sum }_{i = ρ}^{t - 1} [ {f}_{i}^{x} , {f}_{i}^{y} ] , t > ρ \\ {\sum }_{i = t}^{ρ - 1} [ {f}_{i}^{x} , {f}_{i}^{y} ] , t < ρ\end{matrix} \qquad Eq . 8$$

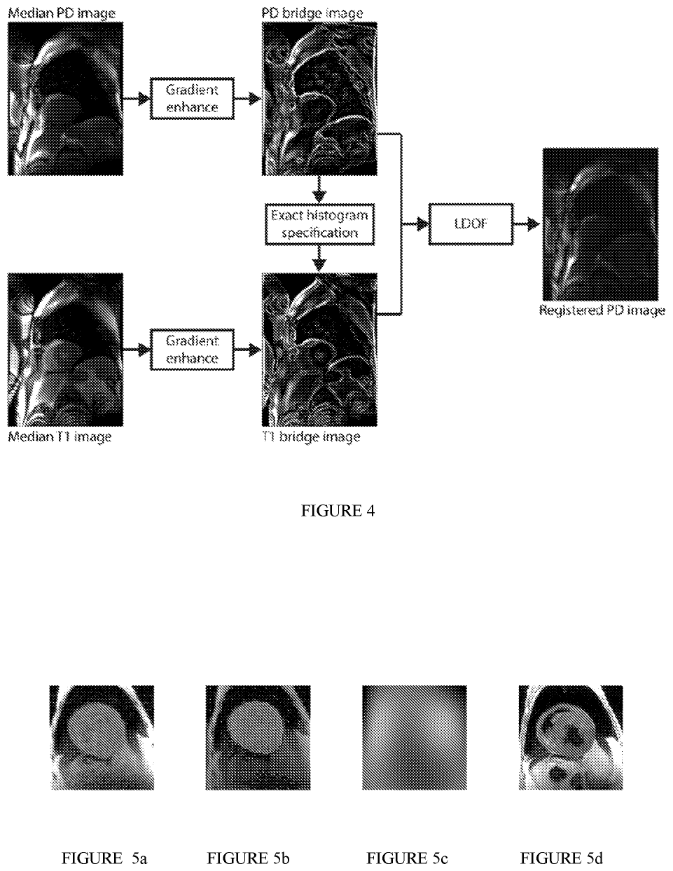

Then PD to non-PD image registration may be performed. Once motion correction for the myocardial or AIF perfusion images is completed, the PD images are aligned to the last PD frame. These images are then registered to the myocardial perfusion and AIF MRI series via bridge images of the PD and T1 frames to match their photometric properties and increase their structural information, as shown in FIG. 4. The T1 bridge image (IbrT1) is constructed by first taking the median (IT1) of the motion-corrected baseline T1 frames leading up to the reference frame ρ as these are not influenced by ventricular contrast dynamics. An edge-enhanced image (IedgeT1) of the IT1 is computed using a weighted combination of its gradient image obtained with the Sobel operator. Similarly, the PD bridge image (IbrPD) is constructed with the median (IPD) and edge-enhanced (IedgePD) images. Next, histogram equalization and un-sharp masking are performed on the IedgeT1 and IedgePD images to further enhance the anatomical boundary gradients. This is followed by exact histogram specification to form the final bridge image pair. The flow field mapping IbrPD to IbrT1 is computed with the LDOF process outlined above using a high gradient consistency parameter γ and a large descriptor matching weight β to emphasize the discrete feature correspondence component. The resulting displacement is finally applied to the motion-corrected PD images to re-register them with the motion-corrected perfusion T1 series.

Image post-processing is then performed. In one embodiment, post-processing of the motion corrected PD, myocardial perfusion and AIF images is limited to matching their photometric profiles with their corresponding raw frames using exact histogram specification. This recovers the original signal intensity distributions that have invariably been altered during the 2D interpolation warping stage. As a result, motion corrected version of the input myocardial perfusion MRI series, motion corrected version of the AIF series and PD images non-linearly registered to the myocardial perfusion MRI series are obtained.

Then intensity correction is performed on the motion corrected version of the input myocardial perfusion MRI series and motion corrected version of the AIF series, as follows, to obtain surface coil bias field, myocardial perfusion series corrected of surface coil bias and AIF series corrected of surface coil bias. It should be understood that the PD images non-linearly registered to the myocardial perfusion and AIF MRI series may be used instead of the motion corrected version of the input myocardial perfusion MRI series.

First, a short-axis stack of PD-weighted MR or baseline myocardial images are used to estimate the signal intensity profile at different imaging locations. Since the proton density of different tissues does not vary much, the signal intensity variation in the image is dominated mostly by the inhomogeneous surface coil reception. A high order polynomial function fitting which combines a hierarchical region weighting scheme is used to approximate the surface coil intensity profile from the PD or baseline myocardial perfusion images. In order to weight the polynomial fitting more to the heart than the surrounding tissues, a higher density of sampling is used inside the heart to further constrain the fitting. The lower density of sampling is used outside the heart and in the background area. The body and background regions are automatically segmented by intensity thresholding. Next, a fitting method such as a fifth-order 2D polynomial least square fitting is used to estimate the signal intensity bias field. The fifth-order polynomial fitting is selected to minimize imperfect surface coil signal intensity fitting at the edges of the epicardium near the lungs since the abrupt transition in proton density at these regions is a high-order form and may be critical to cardiac applications. The estimated signal intensity bias field is then used for correcting the myocardial perfusion and AIF image by dividing these with the estimated intensity bias field.

The output comprises a surface coil bias field such as the one illustrated in FIG. 5c, myocardial perfusion series corrected of surface coil bias such as the image illustrated in FIG. 5d and AIF series corrected of surface coil bias.

In one embodiment, after the surface coil intensity correction, the image series are further adjusted to remove baseline intensity based on baseline myocardial perfusion or AIF images.

Then LV and myocardial signal detection is performed along with timing point detection using the myocardial perfusion series corrected of surface coil bias and the AIF series corrected of surface coil bias in order to obtain signal intensity curves and metrics derived from the signal dynamics and the identification and segmentation of the left ventricle myocardial tissue region.

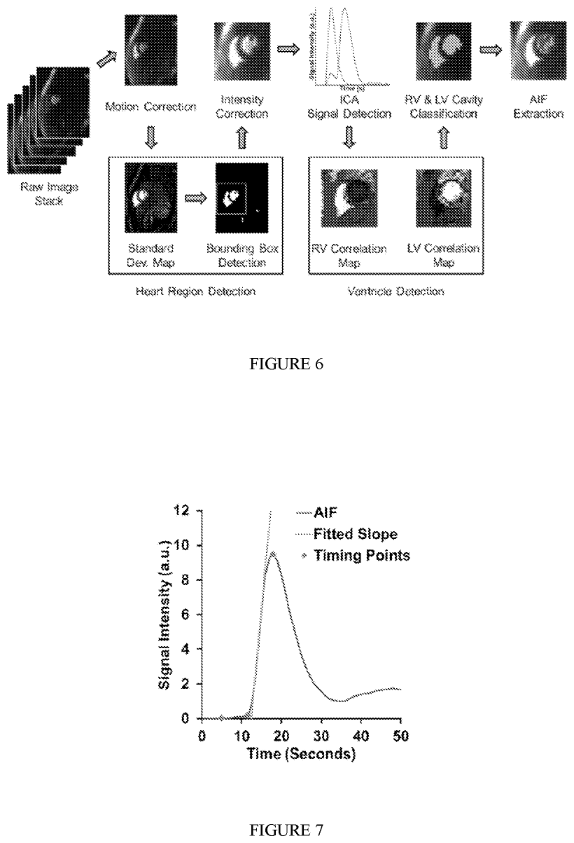

FIG. 6 illustrates one embodiment of the image processing method to measure AIFs from the image series. It should be understood that the same method is applicable to either dedicated AIF or standard myocardial image series.

The heart region is first detected on the myocardial perfusion and AIF images. The location of the heart region, in the form of a bounding box, is determined in order to aid further processing. This may be achieved by identifying regions most indicative of the heart ventricles. Candidate ventricle regions are dynamically thresholded from a pixel-wise standard deviation map of the time series images. This standard deviation map highlights pixels with large intensity changes, such as those caused by contrast agent perfusion, while removing regions that remain constantly bright, such as the chest wall. In one embodiment, this map is thresholded at one standard deviation above the mean for the AIF series, and at two standard deviations above the mean for the myocardial perfusion series to obtain the candidate ventricle regions. It should be understood that the thresholding values, i.e. the one standard deviation for the AIF series and the two standard deviations for the myocardial perfusion series, are exemplary only.

After binarization, regions that do not match the temporal signal characteristics of the ventricles are identified and removed. For example, regions whose intensity increase is less than twice their baseline intensity, thereby indicating minimal contrast enhancement, may be removed. In the same or another example, regions whose peak intensity occurs within the first or last 3 frames of the time series may also be removed.

Next, a similarity check is performed to examine whether each region represents a unique ventricular candidate. Similar regions are grouped as a single ventricular region that may have been split by papillary muscles, image artifacts or slice placement. In one embodiment, similar regions are identified as having average time-signal intensity curves with a cross-correlation coefficient of more than a statistically determined threshold, and whose minimum Euclidean distance is less than the sum of each region's average radius. The final regions are subject to a linear voting scheme to iteratively determine which two candidate regions are most characteristic of the RV and LV cavities based on their time-signal features. Features used for voting classification may include: distance to the image center, distance to previously selected candidate regions, size of each region, signal intensity upslope, peak value (PV), time to peak (TTP), full width at half maximum (FWHM), and/or an M-value which combines the previous three features as shown in Eq. 9:

$$M = \frac{PV}{( TTP * FWHM )} \qquad ( 9 )$$

For each feature, the candidate ventricles are ranked by how well they match typical ventricle characteristics; that is those with larger region size, PV, upslope, M− value, smaller TTP, FWHM, and shorter distances to image center and to the previously selected ventricles. In one embodiment, the ranks are converted to a score of one to N, one being the lowest rank and N, the number of candidate regions, the highest. The feature scores for each region are totaled, and the region with the highest total score is selected as a ventricle region. The second ventricle is selected similarly. The two selected ventricle regions are used to create a bounding box around the heart for subsequent processing.

Then ventricular pixel detection is performed. To further the detection of ventricular blood pool pixels, an independent component analysis (ICA) method is first used to obtain representative time-signal intensity curves from the previously identified ventricular regions. Assuming a mixture of two independent sources of signal (the RV and LV) in all ventricular regions, ICA separates and extracts the two primary time-signals that represent the dynamic contrast of the two ventricles. All of the pixels in the bounding box are classified to the RV, LV or background regions by computing their cross-correlation to the RV and LV time-signals after the ICA process. Pixels with a cross-correlation greater than a statistically determined value are assigned to the matching ventricle; the remaining pixels are then classified as the background region. The RV is identified as being the first region to reach peak intensity, which is followed by the LV region.

The next step consists in selecting LV pixels that are brighter than a default predefined threshold to compute an average intensity value of the blood pool. This step may exclude any papillary muscle pixels that may have been included in the LV region from the previous steps because they do not receive any contrast agent and remain dark. This step may also exclude pixels that contain potential partial volume errors, as these pixels will also be darker than the average LV pixel. This method may closely replicate manual analysis, where the LV cavity is a relatively small but bright region within the heart. The default threshold may be computed from the maximal intensity projection image as the 75th percentile of the maximal intensity range of the LV region. Finally, the signal intensity curve derived from the AIF images is linearly re-sampled at every half second for example to convert the time unit from image frames to seconds, since the perfusion imaging is performed on every R-R interval. This re-sampled curve is referred to as the AIF time-signal intensity curve.

Further, the AIF timing point is calculated. In one embodiment and in order to calculate time-signal characteristics for quantitative perfusion analysis as well as for candidate ventricle region selection, three contrast enhancement time points, i.e. baseline, start of, and peak contrast enhancement points, are derived from the AIF time-signal intensity curve. First, the peak time is detected simply from the peak value of the time-signal intensity curve. The baseline time is determined next as the point of the curve with minimal intensity variation with its neighbors (i.e. the immediately adjacent points) between the beginning of the series and the rising peak (indicated by the point of maximal intensity change before the peak time). Finally, the start time is detected by fitting a line to the rising peak and selecting the point of the curve geometrically closest to the intersection of this fitted line and the baseline intensity. As an example, automatically detected AIF timing points are shown in FIG. 7.

Pixel-wise deconvolution can then be performed to obtain a myocardial blood flow pixel map using the motion corrected myocardial perfusion series, the left ventricle myocardial tissue region segmentation and AIF and myocardial time-signal intensity curves, and optionally the surface coil bias field.

In one embodiment and assuming that the system response of contrast transport within a tissue is linear and stationary, the contrast concentration curve of the tissue may be expressed as a convolution of the arterial input function and an impulse response function. The impulse response function is a probability density function that characterizes contrast transit times through the system. This function, h(t), can be obtained through a reverse process of deconvolution as expressed in Eq. 10. Since deconvolution is sensitive to noise, the shape of h(t) is constrained to a mathematical model. The best parameters describing the model are determined by iterative calculations. This overall process is called model-constrained deconvolution.

$$h ( t ) = \frac{F}{( 1 + exp ( - ( t - τ ) \times k ) )} + I \qquad Eq . 10$$

Eq. 10 proposes a logistic impulse response function where F represents the magnitude of the function, t and k describe the temporal delay length and decay rate, respectively, of h(t) due to dynamically changing contrast concentration.

In one embodiment, this model differs from the commonly used Fermi function by the introduction of an interstitial offset term I. This parameter provides a linear shift of the impulse response function from zero during and after the first pass, which accounts for leakage of the contrast into the interstitial space and the slow clearance relative to the first-pass kinetics. MBF in both pixel-wise and sector-wise analyses are estimated using this model from the LV arterial input and myocardial time-signal intensity curves.

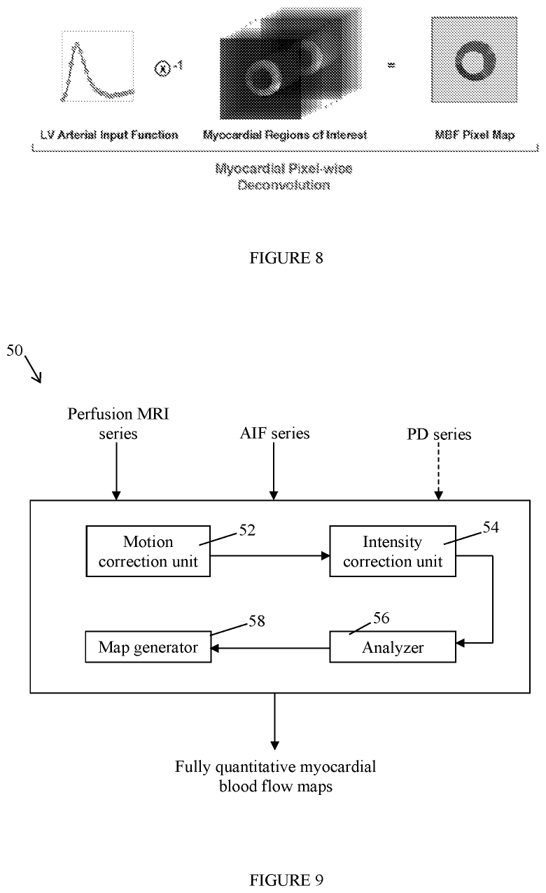

The output is then a myocardial blood flow pixel map as illustrated in FIG. 8.

FIG. 9 illustrates one embodiment of a system 50 for generating fully quantitative myocardial blood flow maps. The system 50 comprises a motion correction unit 52, an intensity correction unit 54, an analyzer 56 and a map generator 58. The system 50 is configured for receiving myocardial perfusion magnetic resonance imaging (MRI) image series and a series of arterial input function (AIF) MRI images. The system 50 may be configured for further receiving PD image series.

The motion correction unit 52 is configured for performing motion correction for the myocardial perfusion MRI images and the AIF MRI images in order to obtain motion corrected myocardial perfusion MRI images and motion corrected AIF images, as described above.

The intensity correction unit 54 is configured for performing intensity correction for the motion corrected myocardial perfusion MRI images and the motion corrected AIF image in order to obtain surface coil intensity corrected MRI images and surface coil intensity corrected AIF images, as described above.

The analyzer 56 is configured for determining time-signal intensity characteristics and segmenting the left ventricle myocardial tissue region using the surface coil intensity corrected MRI images and the surface coil intensity corrected AIF images, as described above.

The map generator 58 is configured for generating myocardial blood flow maps using the motion corrected myocardial perfusion MRI images, the left ventricle myocardial tissue region segmentation and the time-signal intensity characteristics, as described above. The map generator 58 is further configured for outputting the generated myocardial blood flow maps.

In one embodiment, each unit 52-58 is provided with a respective processor, a respective memory and a respective communication unit. In another embodiment, at least two of the units 52-58 may share a same processor, a same memory and/or a same communication unit. For example, the

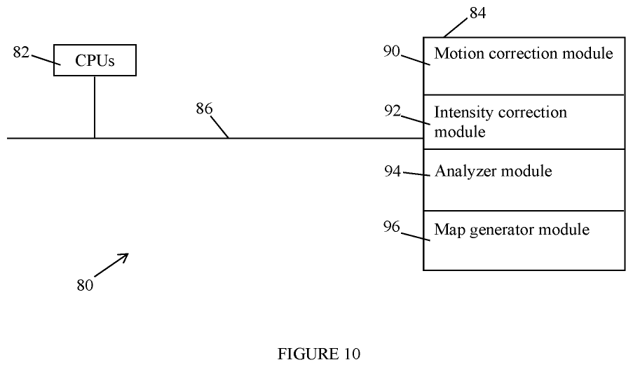

FIG. 10 is a block diagram illustrating an exemplary processing module 80 for executing the steps 12 to 22 of the method 10, in accordance with some embodiments. The processing module 80 typically includes one or more Computer Processing Units (CPUs) and/or Graphic Processing Units (GPUs) 82 for executing modules or programs and/or instructions stored in memory 84 and thereby performing processing operations, memory 84, and one or more communication buses 86 for interconnecting these components. The communication buses 86 optionally include circuitry (sometimes called a chipset) that interconnects and controls communications between system components. The memory 84 includes high-speed random access memory, such as DRAM, SRAM, DDR RANI or other random access solid state memory devices, and may include non-volatile memory, such as one or more magnetic disk storage devices, optical disk storage devices, flash memory devices, or other non-volatile solid state storage devices. The memory 84 optionally includes one or more storage devices remotely located from the CPU(s) 82. The memory 84, or alternately the non-volatile memory device(s) within the memory 84, comprises a non-transitory computer readable storage medium. In some embodiments, the memory 84, or the computer readable storage medium of the memory 84 stores the following programs, modules, and data structures, or a subset thereof:
a motion correction module 90 for performing motion correction for the myocardial perfusion MRI images and the AIF MRI images in order to obtain motion corrected myocardial perfusion MRI images and motion corrected AIF images;
an intensity correction module 92 for performing intensity correction for the motion corrected myocardial perfusion MRI images and the motion corrected AIF image in order to obtain surface coil intensity corrected MRI images and surface coil intensity corrected AIF images;
an analyzer module 94 for determining time-signal intensity characteristics and segmenting the left ventricle myocardial tissue region using the surface coil intensity corrected MRI images and the surface coil intensity corrected AIF images; and a map generator module 96 for generating myocardial blood flow maps using the motion corrected myocardial perfusion MRI images, the left ventricle myocardial tissue region segmentation and the time-signal intensity characteristics.

Each of the above identified elements may be stored in one or more of the previously mentioned memory devices, and corresponds to a set of instructions for performing a function described above. The above identified modules or programs (i.e., sets of instructions) need not be implemented as separate software programs, procedures or modules, and thus various subsets of these modules may be combined or otherwise re-arranged in various embodiments. In some embodiments, the memory 84 may store a subset of the modules and data structures identified above. Furthermore, the memory 84 may store additional modules and data structures not described above.

Although it shows a processing module 80, FIG. 9 is intended more as functional description of the various features which may be present in a management module than as a structural schematic of the embodiments described herein. In practice, and as recognized by those of ordinary skill in the art, items shown separately could be combined and some items could be separated.

In an embodiment in which the generated myocardial blood flow maps comprises stress and rest myocardial blood flow maps, features of interest may be automatically extracted as described below.

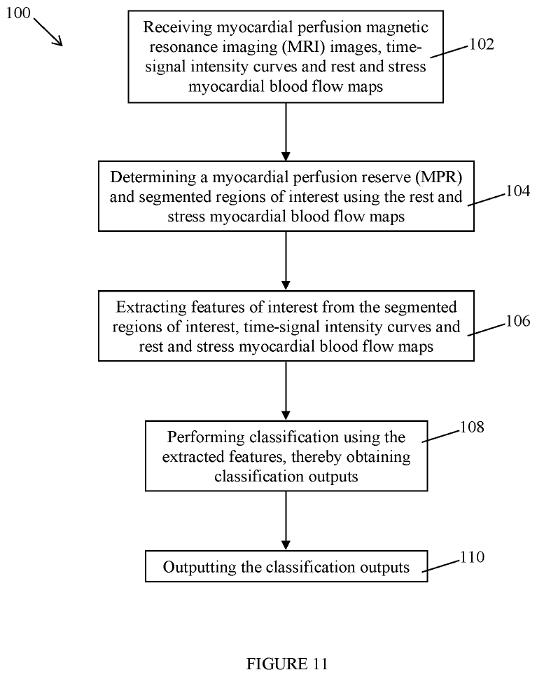

FIG. 11 illustrates one embodiment of a method 100 for automatically detecting and diagnosing heart disease. It should be understood that the method is executed by a computer machine provided with at least one processor or processing unit, a memory and communication means.

At step 102, motion corrected myocardial perfusion magnetic resonance imaging images, time-signal intensity curves and rest and/or stress myocardial blood flow maps are received.

At step 104, a myocardial perfusion reserve (MPR) map and segmented regions of interest encompassing the left ventricle are determined using the rest and stress myocardial blood flow maps.

The automatic perfusion and myocardial blood flow segmentation framework extracts the tissue encompassing the left ventricle using both the motion corrected perfusion images and computed myocardial blood flow maps. The segmentation of this tissue is based on analysis of pixel dynamics undergoing segmentation and may be coupled with region growing or different types of machine learning algorithms that perform spatial segmentation. FIG. 12b illustrates exemplary automatic region-of-interest segmentation of the raw image of FIG. 12a into the right and left ventricles while FIG. 12c illustrates exemplary automatic region-of-interest segmentation of the raw image of FIG. 12a into the left ventricle myocardium.

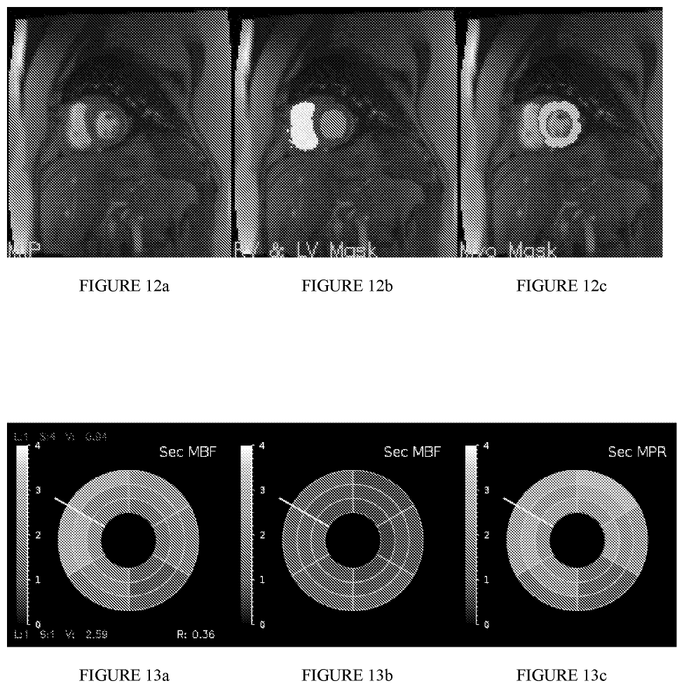

The automatic calculation of the myocardial perfusion reserve (MPR) map is performed by non-rigid registration of the rest myocardial blood flow maps to the stress myocardial blood flow maps. This non-rigid registration is performed using the motion correction module, which is able to perform uni- and multi-modal non-rigid registration. Following the registration of the rest-to-stress myocardial blood flow maps, a pixel-wise division of the stress over rest myocardial blood flow values is performed to compute the MPR. FIG. 13a illustrates an exemplary sector-wise polar plots of myocardial blood flow for a stress series while FIG. 13b illustrates an exemplary sector-wise polar plots of myocardial blood flow for a rest series.

At step 106, features of interest are extracted from the automated quantified and segmented CMR stress and rest myocardial perfusion pixel maps and time-signal intensity curves. These features may include pixel-wise and sector-wise perfusion indices (as illustrated in FIG. 13c which illustrates exemplary sector-wise polar plots of myocardial blood flow for the myocardial perfusion reserve), myocardial blood flow and/or myocardial perfusion reserve estimates measured globally in the entire LV myocardium and/or within specific regions of interest. The measurements may include absolute and relative perfusion that are expressed in physiological units (e.g. ml/g/min, gram, second) or as arbitrary units (e.g. signal intensity, rate, or percentage). The measurements may also include the size, location, texture, pattern, severity, or grading of the lesion (perfusion defect) within the myocardium.

At step 108, classification is performed using the features of interest from the segmented regions of interests of the left ventricle, thereby obtaining classification output. The type of classifier used for classifying the extracted features of interest can be any suitable machine learning engine such as (but not limited to) support vector machine, neural networks, Bayes classifier, k-nearest neighbors, logistic regression model and/or linear discriminant analysis.

In one embodiment, the classification output may comprise:
imaging quality assurance factors such as heart rate, RR interval during ECG gating, vasodilator systematic response and/or signal intensity linearity measurement.
symbols and markers to label the location and size of suspected myocardial lesions in the movies, plots, and maps. Potential image artifacts (e.g. dark rims or motion-correction artifacts) may also be labeled;
anatomical mapping of perfusion defect regions to coronary artery anatomy to predict the location and likelihood of significant stenosis in different coronary arteries;
patterns of perfusion defect maybe useful to predict microvascular rather than coronary artery disease; and/or
an integrated machine generated diagnostic report to summarize global and regional myocardial perfusion, possible myocardial sectors/territories and coronary arteries that may be in jeopardy or require intervention.

At step 110, the classification output is outputted. In one embodiment, the classification output is stored in memory. In the same or another embodiment, the classification output may be displayed on a display unit.

In one embodiment, a diagnostic report may be generated to summarize global and regional myocardial perfusion, possible myocardial sectors/territories and coronary arteries that may be in jeopardy or require intervention.

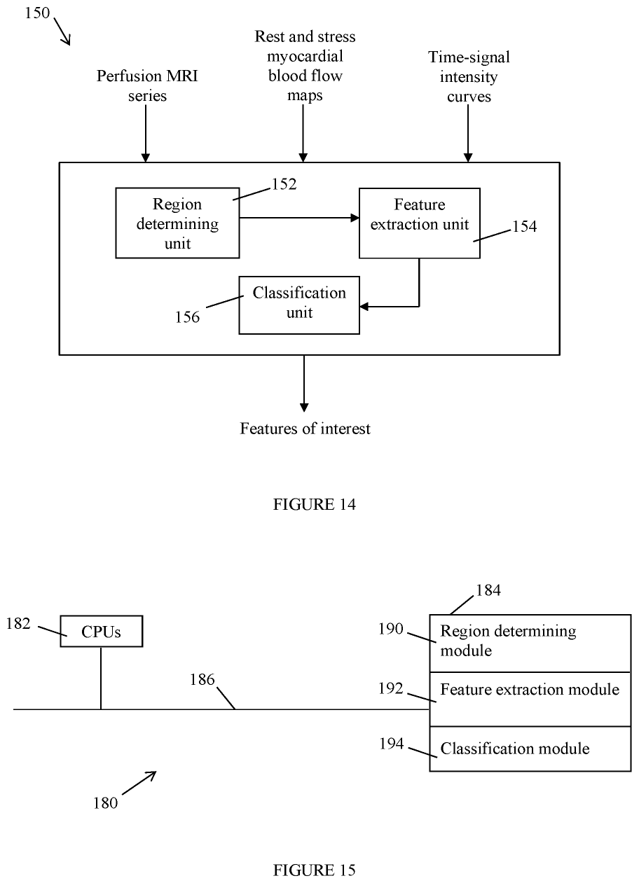

FIG. 14 illustrates one embodiment of a system 150 for analysing myocardial blood flow. The system 150 comprises a region determining unit 152, a feature extraction unit 154 and a classification unit 156.

The system 150 is configured for receiving motion corrected myocardial perfusion magnetic resonance imaging images, time-signal intensity curves and rest and stress myocardial blood flow maps.

The region determining unit 152 is configured for determining a myocardial perfusion reserve (MPR) map and segmented regions of interest of a left ventricle using the rest and stress myocardial blood flow maps, as described above.

The feature extraction unit 154 is configured for extracting features of interest from the segmented regions of interest, the MPR maps, time-signal intensity curves and rest and stress myocardial blood flow maps. The classification unit 156 is configured for classifying the extracted features to obtain a classification output and outputting the classification output to depict normal vs. abnormal myocardial regions and their corresponding coronary territories, as described above.

It should be understood that the system 50 and 150 may be combined together.

FIG. 15 is a block diagram illustrating an exemplary processing module 180 for executing the steps 102 to 108 of the method 100, in accordance with some embodiments. The processing module 180 may be configured for further executing the steps 12 to 20 of the method 10. The processing module 180 typically includes one or more Computer Processing Units (CPUs) and/or Graphic Processing Units (GPUs) 182 for executing modules or programs and/or instructions stored in memory 184 and thereby performing processing operations, memory 184, and one or more communication buses 186 for interconnecting these components. The communication buses 186 optionally include circuitry (sometimes called a chipset) that interconnects and controls communications between system components. The memory 184 includes high-speed random access memory, such as DRAM, SRAM, DDR RAM or other random access solid state memory devices, and may include non-volatile memory, such as one or more magnetic disk storage devices, optical disk storage devices, flash memory devices, or other non-volatile solid state storage devices. The memory 184 optionally includes one or more storage devices remotely located from the CPU(s) 182. The memory 184, or alternately the non-volatile memory device(s) within the memory 184, comprises a non-transitory computer readable storage medium. In some embodiments, the memory 184, or the computer readable storage medium of the memory 184 stores the following programs, modules, and data structures, or a subset thereof:
a region determining module 190 for determining a MPR map and segmented regions of interest of a left ventricle; and
an feature extraction module 192 for extracting features of interest; and
a classification module 194 for classifying the features of interest.

Each of the above identified elements may be stored in one or more of the previously mentioned memory devices, and corresponds to a set of instructions for performing a function described above. The above identified modules or programs (i.e., sets of instructions) need not be implemented as separate software programs, procedures or modules, and thus various subsets of these modules may be combined or otherwise re-arranged in various embodiments. In some embodiments, the memory 84 may store a subset of the modules and data structures identified above. Furthermore, the memory 84 may store additional modules and data structures not described above.

Although it shows a processing module 180, FIG. 12 is intended more as functional description of the various features which may be present in a management module than as a structural schematic of the embodiments described herein. In practice, and as recognized by those of ordinary skill in the art, items shown separately could be combined and some items could be separated.

The embodiments of the invention described above are intended to be exemplary only. The scope of the invention is therefore intended to be limited solely by the scope of the appended claims.

1-51. (canceled)

52. A computer-implemented method for automatically generating a fully quantitative myocardial blood flow map, comprising:
receiving myocardial perfusion magnetic resonance imaging (MRI) images and arterial input function (AIF) MRI images;
correcting a motion of a heart in the myocardial perfusion MRI images and the AIF MRI images, thereby obtaining motion corrected myocardial perfusion MRI images and motion corrected AIF images;
correcting an intensity of the motion corrected myocardial perfusion MRI images and an intensity of the motion corrected AIF images, thereby obtaining surface coil intensity corrected MRI images and surface coil intensity corrected AIF images;
using the surface coil intensity corrected MRI images and the surface coil intensity corrected AIF images, determining time-signal intensity characteristics and segmenting a left ventricle myocardial tissue region;
generating the myocardial blood flow map using the motion corrected myocardial perfusion MRI images, the left ventricle myocardial tissue region segmentation and the time-signal intensity characteristics; and
outputting the myocardial blood flow map.

53. The computer-implemented method of claim 52, wherein said correcting the motion of the heart comprises detecting the motion of the heart in the myocardial perfusion MRI images and the AIF MRI images.

54. The computer-implemented method of claim 53, wherein said detecting the motion of the heart comprises generating a first copy of the myocardial perfusion MRI images and a second copy of the AIF MRI images, and rescaling the first and second copies, thereby obtaining a rescaled copy of myocardial perfusion MRI images and a rescaled copy of AIF MRI images.

55. The computer-implemented method of claim 54, wherein said detecting the motion of the heart comprises performing a non-rigid displacement estimation on the rescaled copy of. myocardial perfusion MRI images and a rescaled copy of AIF MRI images.

56. The computer-implemented method of claim 54, further comprising identifying a reference frame for each one of the rescaled copy of myocardial perfusion MRI images and the rescaled copy of AIF MRI images.

57. The computer-implemented method of claim 56, wherein said correcting the motion of the heart comprises registering the myocardial perfusion MRI images and the AIF MRI images to the reference frame, thereby obtaining the motion corrected myocardial perfusion MRI images and the motion corrected AIF images.

58. The computer-implemented method of claim 57, wherein said registering is performed using an interpolation warping method.

59. The computer-implemented method of claim 57, wherein said correcting the intensity comprises:
estimating a signal intensity bias field; and
correcting the motion corrected myocardial perfusion MRI images and the motion corrected AIF images using a signal intensity bias field, thereby obtaining the surface coil intensity corrected MRI images and the surface coil intensity corrected AIF images.

60. The computer-implemented method of claim 59, wherein said determining time-signal intensity characteristics and segmenting a left ventricle myocardial tissue region comprises identifying a left ventricle and a right ventricle,
said identifying the left ventricle and the right ventricle comprising:
determining candidate ventricle regions within the surface coil intensity corrected MRI images and the surface coil intensity corrected AIF images;
using a similarity check method to regroup particular ones of the candidate ventricle regions to obtain two ventricle regions; and
using a linear voting scheme to assign a first one of the two ventricle regions to the left ventricle and a second one of the two ventricle regions to the right ventricle.

61. A system for automatically generating a fully quantitative myocardial blood flow map, comprising:
a motion correction unit for receiving myocardial perfusion magnetic resonance imaging (MRI) images and arterial input function (AIF) MRI images and correcting a motion of a heart in the myocardial perfusion MRI images and the AIF MRI images in order to obtain motion corrected myocardial perfusion MRI images and motion corrected AIF images;
an intensity correction unit for correcting an intensity of the motion corrected myocardial perfusion MRI images and an intensity of the motion corrected AIF images in order to obtain surface coil intensity corrected MRI images and surface coil intensity corrected AIF images;
an analyzer unit for determining time-signal intensity characteristics and segmenting a left ventricle myocardial tissue region using the surface coil intensity corrected MRI images and the surface coil intensity corrected AIF images; and
a map generator for generating the myocardial blood flow map using the motion corrected myocardial perfusion MRI images, the left ventricle myocardial tissue region segmentation and the time-signal intensity characteristics, and outputting the myocardial blood flow map.

62. The system of claim 61, wherein the motion correction unit is configured for detecting the motion of the heart in the myocardial perfusion MRI images and the AIF MRI images.

63. The system of claim 62, wherein the motion correction unit is configured for generating a first copy of the myocardial perfusion MRI images and a second copy of the AIF MRI images, and rescaling the first and second copies, thereby obtaining a rescaled copy of myocardial perfusion MRI images and a rescaled copy of AIF MRI images.

64. The system of claim 63, wherein the motion correction unit is configured for performing a non-rigid displacement estimation on the rescaled copy of. myocardial perfusion MRI images and a rescaled copy of AIF MRI images.

65. The system of claim 63, wherein the motion correction unit is further configured for identifying a reference frame for each one of the rescaled copy of myocardial perfusion MRI images and the rescaled copy of AIF MRI images.

66. The system of claim 65, wherein the motion correction unit is configured for registering the myocardial perfusion MRI images and the AIF MRI images to the reference frame to obtain the motion corrected myocardial perfusion MRI images and the motion corrected AIF images.

67. The system of claim 66, wherein said registering is performed using an interpolation warping method.

68. The system of claim 57, wherein the intensity correction unit is configured for:
estimating a signal intensity bias field; and
correcting the motion corrected myocardial perfusion MRI images and the motion corrected AIF images using a signal intensity bias field, thereby obtaining the surface coil intensity corrected MRI images and the surface coil intensity corrected AIF images.

69. The system of claim 68, wherein the analyzer unit is configured for identifying a left ventricle and a right ventricle
said identifying the left ventricle and the right ventricle comprising:
determining candidate ventricle regions within the surface coil intensity corrected MRI images and the surface coil intensity corrected AIF images;
using a similarity check method to regroup particular ones of the candidate ventricle regions to obtain two ventricle regions; and
using a linear voting scheme to assign a first one of the two ventricle regions to the left ventricle and a second one of the two ventricle regions to the right ventricle.

70. A computer-implemented method for automatically detecting and diagnosing heart disease, comprising:
receiving myocardial perfusion magnetic resonance imaging (MRI) images, time-signal intensity curves and rest and stress myocardial blood flow maps;
determining a myocardial perfusion reserve (MPR) map and segmented regions of interest of a left ventricle using the rest and stress myocardial blood flow maps and the myocardial perfusion MRI images;
extracting features of interest from the segmented regions of interest, the MPR maps, the time-signal intensity curves and the rest and stress myocardial blood flow maps; and
automatically classifying the features of interest, thereby obtaining a classification output; and
outputting the classification output, the classification output depicting normal versus abnormal myocardial regions and corresponding coronary territories.

71. The computer-implemented method of claim 70, wherein said determining the MPR map is performed using a non-rigid registration of the rest myocardial blood flow map to the stress myocardial blood flow map.

72. The computer-implemented method of claim 70, wherein the classification output comprises at least one of an imaging quality assurance factor, symbols and markers labelling a location and a size of suspected myocardial lesions, an anatomical mapping of perfusion defect regions to a coronary artery anatomy, patterns of perfusion defect and a diagnostic report.

73. The computer-implemented method of claim 72, wherein the imaging quality assurance factor comprises one of a heart rate, an RR interval during electrocardiogram gating, a vasodilator systematic response and a signal intensity linearity measurement.

74. A system for automatically detecting and diagnosing heart disease, comprising:
a region determining unit for receiving myocardial perfusion magnetic resonance imaging (MRI) images, time-signal intensity curves, and rest and stress myocardial blood flow maps, and determining a myocardial perfusion reserve (MPR) map and segmented regions of interest of a left ventricle using the rest and stress myocardial blood flow maps;
a feature extraction unit for extracting features of interest from the segmented regions of interest, the MPR map, the time-signal intensity curves, and the rest and stress myocardial blood flow maps; and
a classification unit for classifying the extracted features of interest to obtain a classification output and outputting the classification output to depict normal versus abnormal myocardial regions and corresponding coronary territories.

75. The system of claim 74, wherein the feature extraction unit is configured for determining the MPR map using a non-rigid registration of the rest myocardial blood flow map to the stress myocardial blood flow map.

76. The system of claim 74, wherein the classification output comprises at least one of an imaging quality assurance factor, symbols and markers labelling a location and a size of suspected myocardial lesions, an anatomical mapping of perfusion defect regions to a coronary artery anatomy, patterns of perfusion defect and a diagnostic report.

77. The system of claim 76, wherein the imaging quality assurance factor comprises one of a heart rate, an RR interval during electrocardiogram gating, a vasodilator systematic response and a signal intensity linearity measurement.
